(19) United States
(12) Patent Application Publication
(10) Pub. No.: US 2012/0078198 A1
(43) Pub. Date: Mar. 29, 2012

(54) VAGINA CLEANING DEVICE

(76) Inventor: Sik Hoong Leong, Singapore (SG)

(21) Appl. No.: 13/376,267

(22) PCT Filed: Jun. 5, 2009
(86) PCT No.: PCT/SG2009/000199

§ 371 Date: Dec. 5, 2011

(51) Int. Cl. A61M 3/02

(52) U.S. Cl. 604/248; 604/246

(57) ABSTRACT

The present invention provides a vagina cleaning device for cleaning vaginas. The vagina cleaning device comprises a body having a first end and a second end, the first end being attachable to a hose for conveying water thereto, inserting nozzle portion being attached to the second end and a flow-pattern regulator engaged in the nozzle portion for regulating the pattern of the water flow through the nozzle portion and into a vagina for cleaning thereof. The cleaning device may comprise a set of devices each having a different flow pattern regulator, or a plurality of different flow pattern regulators may be interchanged in the same cleaning device.

### FIELD OF INVENTION

The present invention relates to a cleaning device, in particular, a vagina cleaning device.

### BACKGROUND

Generally, vaginas of women are not clean and this can be the source of several problems in many women. The pap smear test, a tool to detect cancer of the cervix, can be used to determine whether a vagina is clean or not clean. The pap smear of a clean vagina shows only epithelial cells whereas the pap smear of a vagina, which is not clean, shows inflammatory cells and red blood cells in addition to the epithelial cells.

As mentioned above, vaginas, which are not clean, can be a source of several problems in many women. The problems include unpleasant odour, excessive discharge and infections by virus, bacteria and fungi. However, many of these problems can be addressed or reduced by simply keeping the vaginas clean.

Typically, women try keeping their vaginas clean by spraying water from a shower nozzle thereto during a shower. However, this method of cleaning does not clean the vagina properly, as the water pressure from a shower nozzle is inadequate and the stream of water from the shower nozzle is not properly directed up the vagina. Consequently, only the outside of the vagina and, at best the lower portion of the vagina, is being cleaned since the stream of water is unable to reach the entire length of the vagina.

Besides using the spray from a shower nozzle to clean vaginas, vaginal douches are also used by women to clean vaginas. A vaginal douche is a device used to introduce a stream of liquid into a vagina for medical or hygienic reasons. Vaginal douches typically consist of water mixed with other substances such as vinegar or antiseptic chemicals. Even though vaginal douching adequately cleans the vagina, this method of cleaning is usually used for specific gynaecological problems and cannot be used frequently since frequent usage may result in an imbalance of the pH of the vagina, which may put women at risk for possible vaginal infections such as yeast infections.

Apart from vaginal douches, other devices have been attempted for cleaning vaginas. An example of such a device is disclosed in U.S. Pat. No. 5,401,240. This patent discloses a cleaning device for cleaning vaginas of women. The cleaning device comprises a housing having a bore formed therein, a spout engaged with the housing for insertion into a vagina for guiding water into the vagina, outflow passage means formed in the spout for allowing water to flow out of the vagina, and a valve engaged in the housing for controlling water flowing through the housing and through the spout.

Although this cleaning device cleans vaginas of women, the mechanism for controlling water flow through the housing and the spout are both complex and bulky. Hence, the housing must be large for accommodating the bulky mechanism. Consequently, the cleaning device is very cumbersome which makes it difficult to use and/or manufacture.

Therefore, there is a need for a vagina cleaning device which can be used frequently, and which is capable of thoroughly cleaning the vagina, and which is less cumbersome, and therefore, much easier to use effectively.

### SUMMARY

In accordance with a first embodiment of the invention, there is disclosed a vagina cleaning device which comprises a body having first and second ends. The first end of the housing is attachable to a hose for conveying water from a water source to the body. The vagina cleaning device also comprises an insertable nozzle portion attached to the second end of the housing. The nozzle portion is shaped and dimensioned for inserting and guiding water into the vagina. In this embodiment, each vaginal cleaning device includes one of three flow-pattern regulators positioned within the nozzle portion and each the three flow pattern regulator produce a different flow pattern of which is shaped and dimensioned for the water injected into the vagina depending upon the particular condition of the vagina to be cleaned.

In a second embodiment of the present invention, the vaginal cleaning device is generally in the form of a conventional shower head. However, it further includes an additional and separate conduit within the handle and shower head whereby the shower head also becomes a vaginal cleaning device. In this embodiment, the multiple pattern regulators are removable and interchangeable so as to comprise a dual action cleaner for cleaning both the person's body and the vagina with three different spray patterns.

### BRIEF DESCRIPTION OF THE DRAWINGS

FIG. 1 is a cross-sectional view of the first embodiment of the vaginal cleaning device;

FIG. 2 is a side view of an adaptor for connecting the cleaning device to a conventional water tap or faucet of a conventional sink;

FIG. 3 is a cross-sectional view of the nozzle portion of the cleaner showing one of the flow pattern regulators within the nozzle portion;

FIG. 4 is a cross-sectional view taken along view line 4-4 of FIG. 3;

FIGS. 5 and 6 are cross-sectional views showing second and third types of flow pattern regulators;

FIGS. 5a and 5b are cross-sectional views taken along view lines A and B of FIGS. 5 and 6, respectively.

FIG. 7 is a front elevational view of a second embodiment of the present invention;

FIG. 8 is a side elevational view of the second embodiment shown in FIG. 7;

FIGS. 9-11 are cross-sectional views of alternate forms of flow pattern regulators.

FIG. 12 is a side elevational view of the second embodiment illustrating an alternative form of valve arrangement; and

FIG. 13 is a cross-sectional view taken along view line 13-13 of FIG. 12.

### DETAILED DESCRIPTION

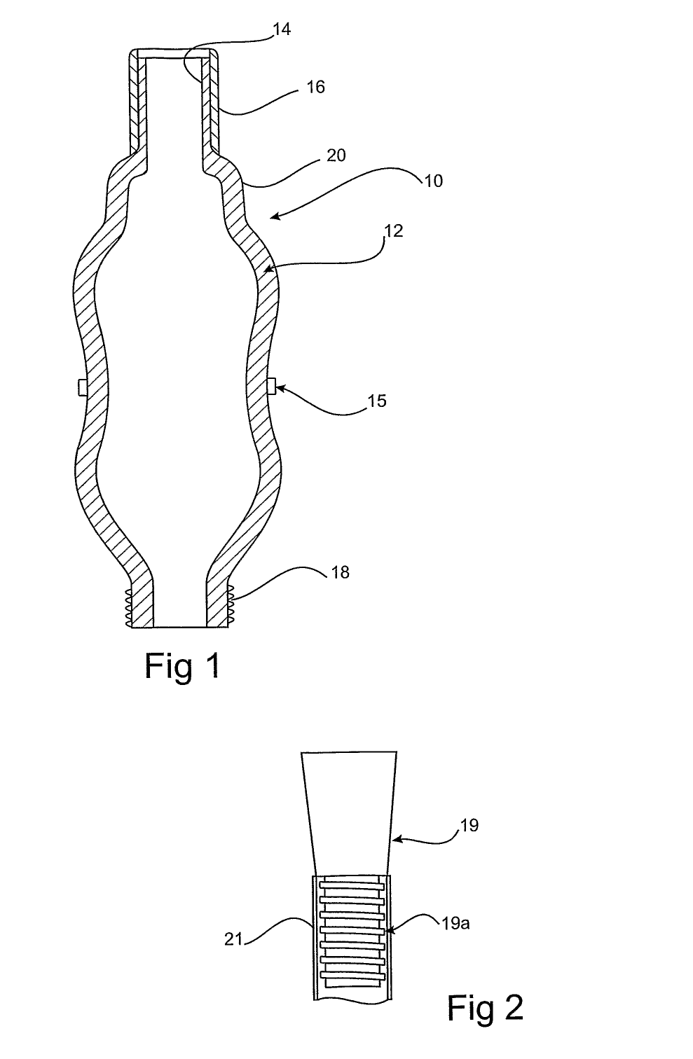

With reference to FIGS. 1 and 2, the vagina cleaning device 10 will be described according to the first embodiment of the invention. The vagina cleaning device 10 comprises a body 12, an insertable nozzle portion 14, and a surrounding sleeve 16 which is composed of a soft material, such as rubber or soft plastic, to prevent any injury to the vagina. The opposite end 18 is preferably threaded for attachment to a flexible tube or hose for conveying water from a water source (not shown) to the body 12. Water is a suitable liquid for cleaning the vagina, as it is non-toxic and has a neutral pH. Hence, it is safe and can be used on a regular basis for cleaning without causing an imbalance of the pH of the vagina. Off course, it will be readily understood that hose 21 may be connected to a water source, such as a bathroom faucet by known connectors, such as for example, an adaptor 19 having flexible sealing ribs 19a, or the hose may be connected by a known treaded connector to the shower head outlet pipe when the conventional shower head is unscrewed and removed.

With respect to dimensions, nozzle portion 14 is preferably in the order of two to three centimetres, and it will be noted that end 20 of the body 20 is shaped and is made larger than the nozzle portion for preventing over insertion of the nozzle portion 14 into the vagina. The preferred length of the entire vagina cleaning device 10 is approximately thirteen to fourteen centimetres, and the preferred diameter of the largest portion of the vagina cleaning device 10 is approximately five centimetres.

However, the size of the vagina cleaning device 10 can be increased or decreased to suit women with different hand sizes. For example, the size of the vagina cleaning device 10 can be reduced proportionately by ten percent (10%) to cater to women with small hand sizes. The size of the vagina cleaning device 10 can also be increased proportionately by 10% to cater to women with large hand sizes. It is also understood that, preferably, colored elastic bands 15 are provided such that multiple users in the same household may identify their personal cleaning device.

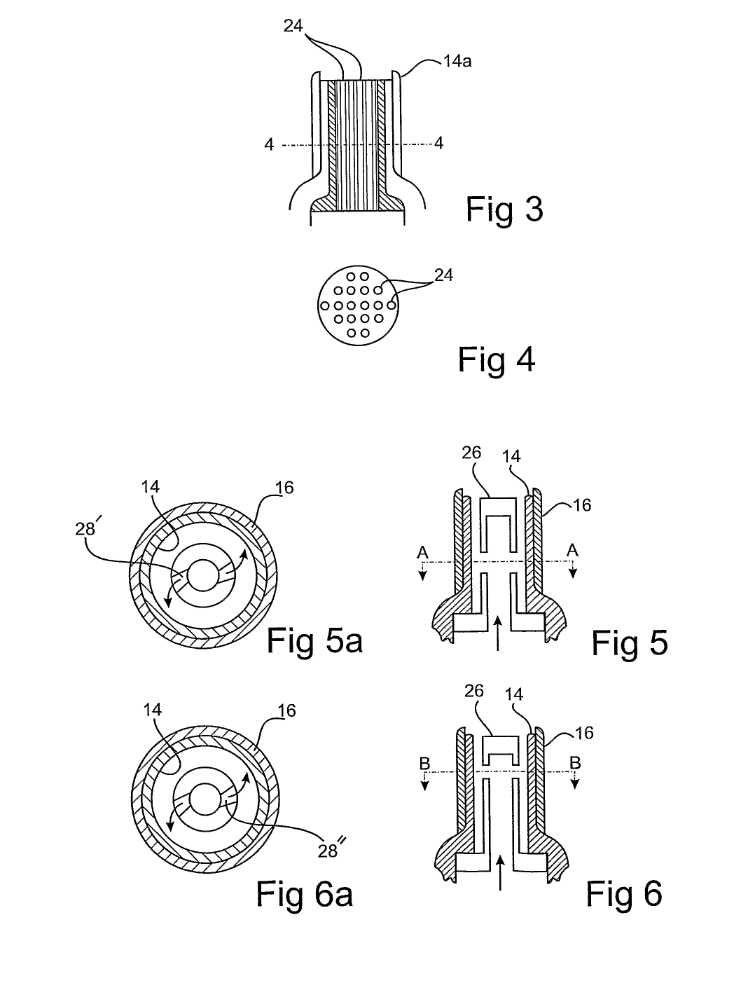

With respect to the multiple flow pattern regulators, in the cleaning device shown in FIG. 1, the flow-pattern regulator comprises a straight and open nozzle 14 which provides a solid flush stream of water flow therethrough and into the vagina. The flush stream of water flow is suitable for daily cleaning of the vagina and is intended to comprise one cleaner of a set of three cleaners each with a different flown pattern regulators such as further shown in FIGS. 4-6 which are described as follows. As shown in FIGS. 4-6, the vagina cleaning devices comprises flow-pattern regulators which are secured in a set of three devices. The regulators of FIGS. 3-4 is provided with a plurality of individual passages 24 which produce a spray pattern of the water. Such a spray-type water flow is gentler as compared to the flush stream of water flow provided by the open nozzle 14. Hence, the spray-type water flow is suitable for use when there is increased sensitivity in the vagina. The sensitivity in the vagina can be increased during menstruation, or when there is a vaginal infection.

The third flow pattern regulator is shown in FIGS. 5-6 which is shaped and dimensioned for providing a spiral water flow therethrough and into the vagina. This regulator includes a closed end tube 26 positioned centrally of nozzle 14. Tube 26 includes upper and lower tangentially extending slots 28′ and 28″ such that the water forced into the tube creates a spiral or vortex motion of the water as it enters the vagina. This spiral water flow produces more vigorous cleaning action as compared to the flush stream of water flow and the spray pattern of the previously described flow regulators. This spiral water flow is suitable for use when thorough cleaning of the vagina is required, for example, after sexual intercourse.

The nozzle portion 14 is made of flexible material, preferably soft plastics. Soft plastics make the nozzle portion 14 soft and smooth, and hence, make insertion of the inserting portion 14 safe and comfortable for a user. The housing 12 is also made of flexible material, preferably soft plastics, and is preferably molded of one piece plastic.

In operation, the vagina cleaning device 10 is held in an upright position with the nozzle portion 14 and sleeve 16 facing upwardly, and with the end 18 of the body 12 facing downwardly with the hose attached to the end 18. Once the upright position of the vagina cleaning device 10 is maintained, water from the water source is conveyed to the body 12 via the hose. The water then flows through the body 12 to the nozzle portion 14. Preferably, the user adjusts the stream of water that flows out of the nozzle portion 14 to reach a height of approximately ten centimetres above the nozzle before inserting the nozzle into a vagina for cleaning thereof.

Since each of the flow-pattern regulators are simple and not bulky, the flow-pattern regulators can be positioned in the narrowest portion of the vagina cleaning device which is the nozzle portion 14. Consequently, the vagina cleaning device is not bulky and thus makes it easy to use and manufacture.

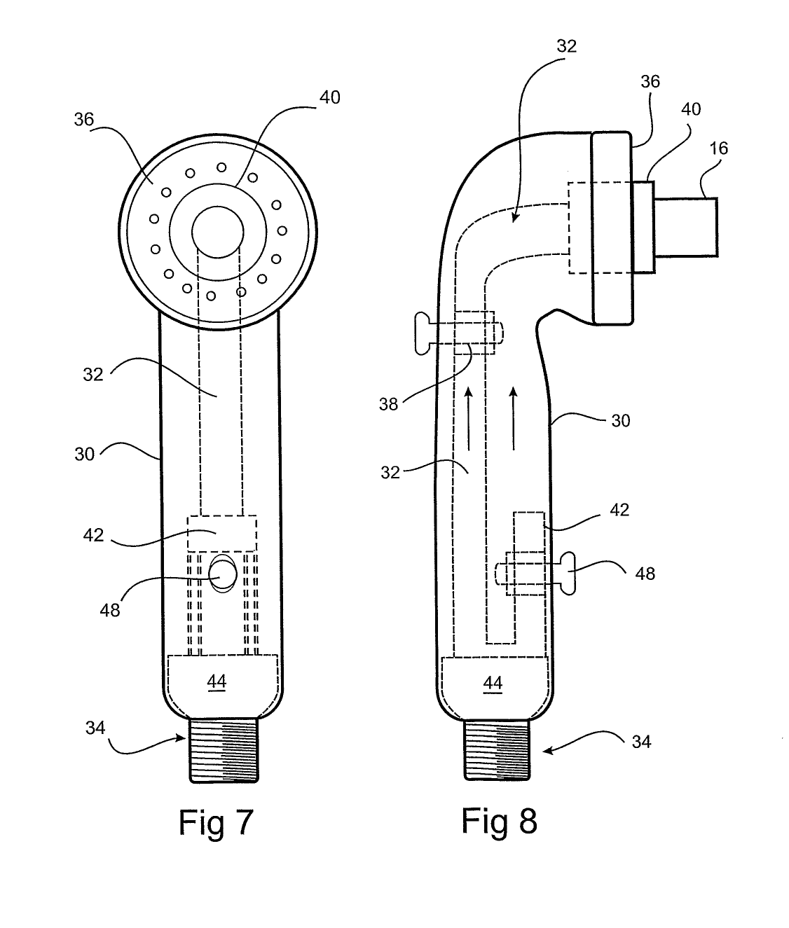

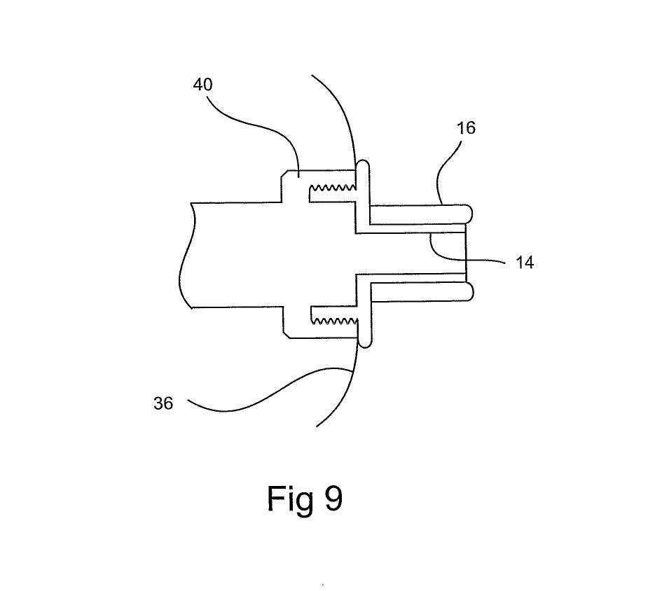

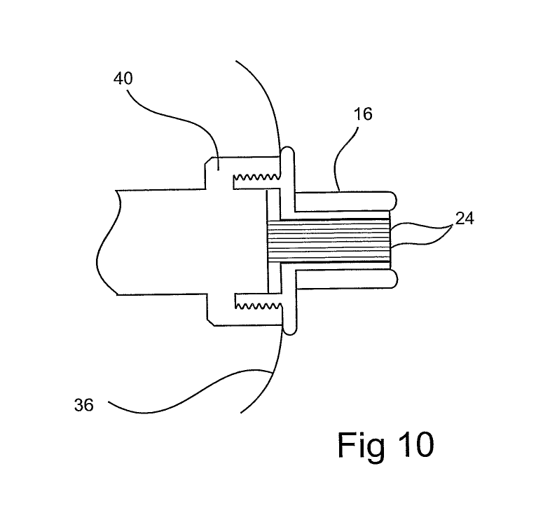

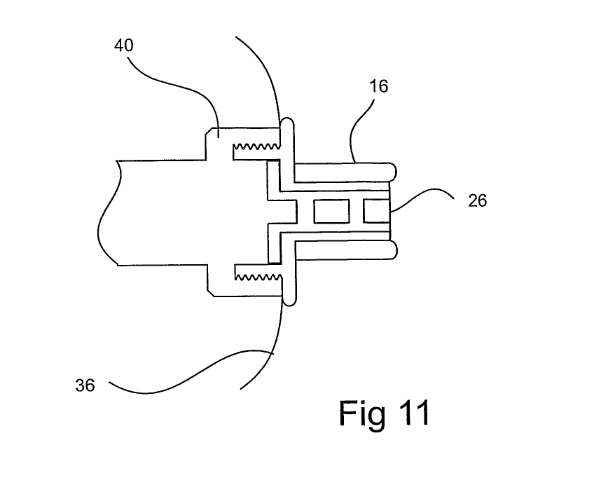

The second embodiment of the present invention will now be described with reference to FIGS. 7-8. In this embodiment, the vaginal cleaning device is generally in the shape of a conventional, hand-held shower head which may be connected by a flexible tube or hose to a source of pressurized water such as the outlet pipe of a conventional shower or bathroom faucet as previously described. Accordingly, this device is preferably shaped as a hollow body 30 within which there extends an internal tube or conduit 32 leading from the inlet end 34 to the head portion 36. A control valve 38, shown as a push-button type valve, is positioned along the length of internal tube 32 so as to open or close the flow of water through tube 32. Internal tube 32 terminates in an enlarged housing 40 forming a chamber for receiving, one at a time, each of the flow pattern regulators as previously described and as shown in FIGS. 9-11.

Therefore, in this embodiment, there is a single unit as opposed to the set of separate units of the previously described embodiment.

As further shown most clearly in FIG. 8, a second shorter tube 42 extends from the plenum chamber 44 formed by the hollow inlet end of the hollow body 30. Tube 42 contains a second control valve 48 whereby the flow of water from plenum 44 may be turned on or off such that the shower head 36 may be turned on or off. Accordingly, it will be understood the embodiment of FIGS. 7-8 may operate in three different modes. That is, with valve 48 open and valve 38 closed, the unit operates as a conventional shower. Secondly, when valve 48 is closed and valve 38 is open, the unit operates as a vaginal cleaning device as the water flows out of nozzle 16 through one or other of the three flow pattern regulators described above and shown in FIGS. 9-11. Thirdly, when both of valves 38 and 48 are open, the unit functions both as a conventional shower head to wash the external portions of the vagina, and also as a vaginal cleansing device to clean the vagina internally.

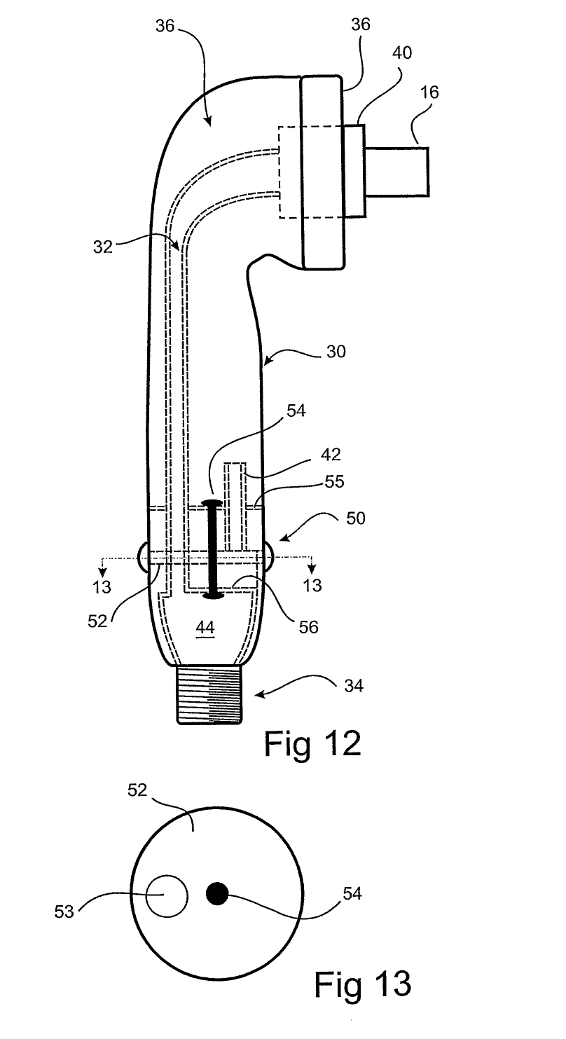

A variation of the embodiment of FIGS. 7 and 8 is illustrated in FIGS. 12 and 13. This variation generally operates in the same manner as that in FIGS. 7-8, and the same elements are designated by the same reference numerals. The difference in this variation is that the operation of valves 38 and 48 are combined in a single, simplified rotary disc valve 50. Valve 50 comprises a valve disc 52, having an aperture 53, which pivots or rotates about a central pin or shaft 54 secured in internal flanges 55 and 56. Thus, it will be apparent that when disc 52 is rotated such that aperture 53 comes into alignment with internal conduct 32, the water flows through conduit 32 to nozzle 16 for use as the vaginal cleaning device as previously described. On the other hand, when disc 52 is turned such that aperture 53 is aligned with tube 42, then the water flows to the shower head such that the unit functions as a conventional shower head.

From the forgoing description of two embodiment of the invention, it will be apparent that the present invention provides a substantially improved vaginal cleaning device which is extremely effective, small in size, simple to operate, and which may be manufactured at relatively low cost. Of course, it will be apparent that many changes and variations of the present invention will become apparent to those skilled in the art of shower heads and other devices for controlling the flow of water to clean various parts of the body. Therefore, it is to be understood that the foregoing description of two preferred embodiments is intended to be purely illustrative of the principles of the invention, rather than exhaustive thereof, and that the present invention is not intended to be limited other than as expressly set forth in the following claims.

1. (canceled)

2. (canceled)

3. (canceled)

4. (canceled)

5. (canceled)

6. (canceled)

7. A vaginal cleaning device comprising:
a hollow body portion connected to a spray head at one end and to a connector at the opposite end for connection to a water supply;
a nozzle portion for insertion into a vagina extending outwardly of the spray head;
a first fluid conduit within the hollow body portion for supplying water from the connector to the nozzle portion;
a second fluid conduit within the hollow body portion for supplying water from the connector to the spray head; and
at least one valve element for selectively controlling a flow of water through the first and second fluid conduits to the nozzle and to the spray head.

8. The vaginal cleaning device of claim 7 including a plurality of different shaped spray pattern regulators of a size shape such as to be interchangeably connected to the nozzle portion for producing different spray patterns of water into the vagina.

9. The vaginal cleaning device of claim 7 wherein a first valve element is positioned in the first fluid conduit, and a second valve element is positioned in the second fluid conduit.

10. The vaginal cleaning device of claim 7 wherein the valve element comprises a single rotary valve connected to each of the first and second fluid conduits whereby the flow to each of the conduits is controlled by the single rotary valve.
